(19) United States
(12) Patent Application Publication
(10) Pub. No.: US 2025/0017608 A1
(43) Pub. Date: Jan. 16, 2025

(54) SEQUENCING AND STRATEGIES FOR ENHANCING INTRAVASCULAR THROMBUS DISRUPTION WITH PHASE-CHANGE CAVITATION ENHANCING AGENTS

(71) Applicants: NORTH CAROLINA STATE UNIVERSITY, Raleigh, NC (US); THE UNIVERSITY OF NORTH CAROLINA AT CHAPEL HILL, Chapel Hill, NC (US); THE REGENTS OF THE UNIVERSITY OF MICHIGAN, Ann Arbor, MI (US)

(72) Inventors: Paul Alexander Dayton, Carrboro, NC (US); Jinwook KIM, Chapel Hill, NC (US); Xiaoning Jiang, Cary, NC (US); Bohua Zhang, Raleigh, NC (US); Huaiyu Wu, Raleigh, NC (US); Zhen Xu, Ann Arbor, MI (US)

(21) Appl. No.: 18/715,639

(22) PCT Filed: Dec. 1, 2022
(86) PCT No.: PCT/US2022/051508

§ 371 Date: May 31, 2024

Related U.S. Application Data
(60) Provisional application No. 63/284,902, filed on Dec. 1, 2021.

(51) Int. Cl. A61B 17/22

(52) U.S. Cl. CPC A61B 17/22012

(57) ABSTRACT

A method for disrupting a blood clot with ultrasound and at least one cavitation enhancing agent includes administering at least one cavitation enhancing agent into a blood vessel of a subject. The method further includes monitoring the at least one cavitation enhancing agent to determine when at least a portion of the at least one cavitation enhancing agent has reached a blood clot. The method further includes controlling application of ultrasound energy to the at least one cavitation enhancing agent within the blood vessel of the subject such that, during a first time period, cavitation-enhancing ultrasound energy is not applied to the at least one cavitation enhancing agent within the blood vessel and, during a second time period after the first time period, cavitation enhancing ultrasound energy is applied to the at least one cavitation enhancing agent within the blood vessel.

### PRIORITY CLAIM

This application claims the priority benefit of U.S. Provisional Patent Application Ser. No. 63/284,902, filed Dec. 1, 2021, the disclosure of which is incorporated herein by reference in its entirety.

### GOVERNMENT INTEREST

This invention was made with government support under Grant No. HL141967 awarded by the National Institutes of Health. The government has certain rights in the invention.

### TECHNICAL FIELD

The subject matter described herein relates to intravascular thrombus disruption using ultrasound energy in combination with a phase-change cavitation enhancing agent.

### BACKGROUND

The application of ultrasound in the presence of one or more cavitation enhancing agents can be used to disrupt blood clots/thrombus present within blood vessels. The term “phase-change cavitation enhancing agent” is used herein to refer to a solution including one or more particles, each of which has a core consisting at least partially of a material that is in a liquid phase. An example of a phase-change cavitation enhancing agent is a solution of encapsulated perfluorocarbon nanodroplets. The term “cavitation enhancing agent” is intended to refer to a solution of phase-change cavitation enhancing agents as well as microbubbles that are capable of enhancing cavitation and disrupting a blood clot.

Phase-change cavitation enhancing agents are unique in that they are not acoustically active until they are triggered with sufficient energy to cause a phase transition. Here we disclose strategies and sequencing of ultrasound pulsing and administration of the cavitation enhancing agents to provide improved thrombus disruption.

### SUMMARY

A method for disrupting a blood clot with ultrasound and at least one cavitation enhancing agent includes administering at least one cavitation enhancing agent into a blood vessel of a subject. The method further includes monitoring the at least one cavitation enhancing agent to determine when at least a portion of the at least one cavitation enhancing agent has reached a blood clot. The method further includes controlling application of ultrasound energy to the at least one cavitation enhancing agent within the blood vessel of the subject such that, during a first time period, cavitation-enhancing ultrasound energy is not applied to the at least one cavitation enhancing agent within the blood vessel and, during a second time period after the first time period, cavitation enhancing ultrasound energy is applied to the at least one cavitation enhancing agent within the blood vessel, wherein the first time period is based on results of the monitoring to determine when the at least a portion of the at least one cavitation enhancing agent reaches the blood clot and the second time period is based on a time for the at least one cavitation enhancing agent to initiate disruption of the blood clot.

According to another aspect of the subject matter described herein, administering the at least one cavitation enhancing agent includes administering a mixture of nanodroplets and microbubbles of the at least one cavitation enhancing agent into the blood vessel.

According to another aspect of the subject matter described herein, administering the at least one cavitation enhancing agent includes separately administering nanodroplets and microbubbles of the at least one cavitation enhancing agent into the blood vessel.

According to another aspect of the subject matter described herein, monitoring the at least one cavitation enhancing agent includes transmitting ultrasound energy into the blood vessel, detecting ultrasound energy scattered by the at least one cavitation enhancing agent, and determining a location of the at least one cavitation enhancing agent within the blood vessel based on the detected ultrasound energy.

According to another aspect of the subject matter described herein, transmitting ultrasound energy into the blood vessel includes transmitting ultrasound energy at a power level selected so as not to cause a phase change in particles of the at least one cavitation enhancing agent.

According to another aspect of the subject matter described herein, transmitting ultrasound energy into the blood vessel includes transmitting ultrasound energy at a power level selected to cause a phase change in particles of the at least one cavitation enhancing agent from a liquid phase to a gaseous phase and form bubbles and to cause the bubbles to burst and wherein monitoring the at least one cavitation enhancing agent includes detecting ultrasound energy scattered by the bursting of the bubbles.

According to another aspect of the subject matter described herein, the first time period ends if a detected location of the bursting of the bubbles is determined to correspond to a location of the blood clot.

According to another aspect of the subject matter described herein, the method of disrupting a clot with ultrasound and at least one cavitation enhancing agent includes ceasing transmission of the ultrasound energy and extending the first time period if a detected location of the bursting of the bubbles is determined to be upstream from a location of the blood clot.

According to another aspect of the subject matter described herein, the first time period is estimated based on an estimated flow rate of the at least one cavitation enhancing agent through the blood vessel in addition to the monitoring.

According to another aspect of the subject matter described herein, administering the at least one cavitation enhancing agent into the blood vessel includes administering the at least one cavitation enhancing agent via a hollow lumen of a catheter and the first time period is based on an estimated flow rate of the at least one cavitation enhancing agent through the hollow lumen.

According to another aspect of the subject matter described herein, the at least one cavitation enhancing agent comprises nanodroplets, and the second time period is based on a time for a portion of the nanodroplets to convert to microbubbles and oscillate in diameter.

According to another aspect of the subject matter described herein, controlling the application of the ultrasound energy comprises applying the cavitation enhancing ultrasound energy to the at least one cavitation enhancing agent for a third time period after the second time period, wherein the third time period is based on a time for a portion of the microbubbles to fragment.

According to another aspect of the subject matter described herein, the method of disrupting a blood clot with ultrasound and at least one phase change cavitation enhancing agent includes monitoring results of application of the cavitation enhancing ultrasound energy to the at least one cavitation enhancing agent and wherein controlling application of the ultrasound energy includes controlling the application based on the results.

According to another aspect of the subject matter described herein, monitoring results of the application of the cavitation enhancing ultrasound energy includes using an ultrasound transducer to monitor ultrasound energy scattered by the at least one cavitation enhancing agent proximal to the blood clot, generating an image of the scattered ultrasound energy, and displaying the image to a user.

According to another aspect of the subject matter described herein, monitoring results of the application of the cavitation enhancing ultrasound energy includes generating a feedback control signal based on ultrasound energy scattered by the at least one cavitation enhancing agent and automatically controlling at least one of the administering of the at least one cavitation enhancing agent and the application of the cavitation enhancing ultrasound energy using the feedback control signal.

According to another aspect of the subject matter described herein, a system for disrupting a blood clot with ultrasound and at least one cavitation enhancing agent is provided. The system includes at least one cavitation enhancing agent capable of being administered into a blood vessel of a subject. The system further includes a receiver for monitoring the at least one cavitation enhancing agent to determine when at least a portion of the at least one cavitation enhancing agent has reached a blood clot. The system further includes a timing controller for controlling application of ultrasound energy to the at least one cavitation enhancing agent within the blood vessel of the subject such that, during a first time period, cavitation-enhancing ultrasound energy is not applied to the at least one cavitation enhancing agent within the blood vessel and, during a second time period after the first time period, cavitation enhancing ultrasound energy is applied to the at least one cavitation enhancing agent within the blood vessel, wherein the first time period is based on results of the monitoring to determine when the at least a portion of the at least one cavitation enhancing agent reaches the blood clot and the second time period is based on a time for the at least one cavitation enhancing agent to initiate disruption of the blood clot.

According to another aspect of the subject matter described herein, the at least one cavitation enhancing agent comprises a mixture of nanodroplets and microbubbles.

According to another aspect of the subject matter described herein, the at least one cavitation enhancing agent comprises nanodroplets and microbubbles separately administerable into the blood vessel.

According to another aspect of the subject matter described herein, the system for disrupting a blood clot with ultrasound and at least one cavitation enhancing agent includes an ultrasound transducer integrated with or separate from the receiver for transmitting ultrasound energy into the blood vessel, wherein the receiver is configured to detect ultrasound energy scattered by the at least one cavitation enhancing agent and the timing controller is configured to determine a location of the at least one cavitation enhancing agent within the blood vessel based on the detected ultrasound energy.

According to another aspect of the subject matter described herein, the ultrasound transducer is configured to transmit the ultrasound energy at a power level selected so as not to cause a phase change in particles of the at least one cavitation enhancing agent.

According to another aspect of the subject matter described herein, the ultrasound transducer is configured to transmit the ultrasound energy at a power level selected to cause a phase change in particles of the at least one cavitation enhancing agent from a liquid phase to a gaseous phase and form bubbles and to cause the bubbles to burst and the receiver is configured to detect the ultrasound energy scattered by the bursting of the bubbles.

According to another aspect of the subject matter described herein, the first time period ends if a detected location of the bursting of the bubbles is determined to correspond to a location of the blood clot.

According to another aspect of the subject matter described herein, the timing controller is configured to cease transmission of the ultrasound energy and extend the first time period if a detected location of the bursting of the bubbles is determined to be upstream from a location of the blood clot. According to another aspect of the subject matter described herein, the system for disrupting a blood clot with ultrasound and at least one cavitation enhancing agent includes a catheter having a hollow lumen through which the at least one cavitation enhancing agent is administerable and the first time period is based on an estimated flow rate of the at least one cavitation enhancing agent through the hollow lumen.

According to another aspect of the subject matter described herein, the at least one cavitation enhancing agent comprises nanodroplets and the second time period is based on a time for a portion of the nanodroplets to convert to microbubbles and oscillate in diameter.

According to another aspect of the subject matter described herein, the timing controller is configured to control the application of the ultrasound energy by applying the cavitation enhancing ultrasound energy to the at least one cavitation enhancing agent for a third time period after the second time period, wherein the third time period is based on a time for a portion of the microbubbles to fragment.

According to another aspect of the subject matter described herein, the receiver is configured to monitor ultrasound energy scattered by the at least one cavitation enhancing agent proximal to the blood clot, generate an image of the scattered ultrasound energy, and display the image to a user.

According to another aspect of the subject matter described herein, the receiver is configured to generate a feedback control signal based on ultrasound energy scattered by the at least one cavitation enhancing agent and the timing controller is configured to automatically control at least one of the administering of the at least one cavitation enhancing agent and the application of the cavitation enhancing ultrasound energy using the feedback control signal.

According to another aspect of the subject matter described herein, a system for implementing any of the methods described or claimed herein is provided.

According to another aspect of the subject matter described herein, a non-transitory computer readable medium having stored thereon computer-executable instructions that when executed by a processor of a computer control the computer to implement any of the methods described or claimed herein is provided.

A method of disrupting a blood clot with ultrasound and at least one phase-change cavitation enhancing agent includes administering at least one cavitation enhancing agent into a blood vessel of a subject. The method further includes controlling application of ultrasound energy to the at least one cavitation enhancing agent within the blood vessel of the subject such that, during a first time period, cavitation enhancing ultrasound energy is not applied to the at least one cavitation enhancing agent within the blood vessel and, during a second time period after the first time period, cavitation enhancing ultrasound energy is applied to the at least one cavitation enhancing agent within the blood vessel, wherein the first time period is based on a time for the at least one cavitation enhancing agent to reach and penetrate a blood clot and the second time period is based on a time for the at least one cavitation enhancing agent initiate disruption of the blood clot.

The cavitation enhancing ultrasound energy applied during the second time period may be energy of a sufficient level to cause the at least one cavitation enhancing agent to cavitate and eventually fracture, causing clot disruption.

The subject matter described herein can be implemented in software in combination with hardware and/or firmware. For example, the subject matter described herein can be implemented in software executed by a processor. In one exemplary implementation, the subject matter described herein can be implemented using a non-transitory computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

Exemplary implementations of the subject matter described herein will now be explained with reference to the accompanying drawings, of which:

FIG. 1 is a block diagram illustrating exemplary components of a system for sequencing of ultrasound pulsing and administration of cavitation enhancing agents to provide improved thrombus disruption;

FIG. 2 is a graph illustrating timing for sequencing of ultrasound pulsing and administration of cavitation enhancing agents to provide improved thrombus disruption;

FIG. 3 is a diagram illustrating an intravascular ultrasound probe used in combination with cavitation enhancing agents to disrupt a thrombus within a blood vessel; and

FIG. 4 is a flow chart illustrating an exemplary process for disrupting a blood clot with ultrasound and at least one cavitation enhancing agent.

### DETAILED DESCRIPTION

The subject matter described herein is based on the application of a device that provides ultrasound energy, a device that controls the timing and amplitude of the ultrasound energy, and a means to introduce the cavitation enhancing agent into the vasculature of a subject, such as a human or a non-human mammal.

Particles that form a phase-change cavitation enhancing agent, such as phase-shift microbubbles, phase-shift nanobubbles, a phase-shift nanoemulsion, or nanodroplets, convert from a liquid (droplet) to a gas (microbubble) with a pulse of ultrasound energy of sufficient amplitude, frequency, and duration. In one implementation, low-boiling point phase-change cavitation enhancing agents are utilized. Particles of a low boiling-point phase-change cavitation enhancing agent each comprise a core of a liquid perfluorocarbon with a boiling point of less than 25° C. at atmospheric pressure, and a stabilizing shell such as a lipid, polymer, or protein. Particles of a low boiling point phase-change cavitation enhancing agent used with the subject matter described herein may contain perfluorocarbons, such as decafluorobutane or octofluoropropane, in their cores. A low boiling point phase-change cavitation enhancing agent can be activated with ultrasound amplitudes that are lower than phase-change cavitation enhancing agents made with perfluorocarbons which are a liquid at 25° C. or greater at atmospheric pressure and so may be desirable for the described application to avoid bioeffects to healthy tissue caused by higher ultrasound energies.

In one implementation, particles of a phase-change cavitation enhancing agent are made into a metastable form by cooling below 0° C. and subjected to a pressure of greater than one atmosphere, prior to being administered into a blood vessel.

In one implementation, particles of a phase-change cavitation enhancing agent are made into a metastable form by cooling below 0° C. and processed in a microfluidizer or homogenizer, prior to being administered into a blood vessel.

Once a particle of a phase-change cavitation enhancing agent has converted to a microbubble, additional ultrasound energy at a sufficient amplitude, frequency, and duration can cause the microbubble to oscillate and fragment. Once a microbubble fragments and dissolves, it is no longer acoustically active.

When a microbubble is adjacent to the surface of a clot and it is made to oscillate and/or fragment, the mechanical action of the bubble vibration can break up the clot. This effect is even more pronounced if the bubble is formed within the clot.

Microbubbles, typically on the order of 1 micron or greater, have a difficult time penetrating into a clot. In contrast, particles of a phase-change cavitation enhancing agent of diameter of less than a micron (typically less than 0.3 microns) can penetrate into and generate microbubbles inside a clot.

Thus, there is a desire for the particles of the phase-change cavitation enhancing agent to at least be positioned within close proximity (<2 mm) to the surface of a blood clot, and ideally, to penetrate the clot prior to forming the microbubble, or prior to the microbubble oscillating and fragmenting. If the microbubble fragments before getting close to or penetrating the clot, the cavitation will be less effective in the clot disruption since the microbubble is not within or close to the clot. Therefore, the sequence of administration of the ultrasound in relation to the administration of the cavitation enhancing agent may be important to achieving optimal clot disruption.

The phase-change cavitation enhancing agent can be administered into the peripheral vasculature of the patient or more directly to the clot through a catheter with a hollow lumen designed to carry the phase-change cavitation enhancing agent. In one implementation, a pump is utilized to provide a constant flow of the phase-change cavitation enhancing agent into the patient's vasculature.

The phase-change cavitation enhancing agent can be “primed” to be more readily be activated (can be activated at a lower acoustic amplitude) if a plurality of microbubbles is mixed with the phase-change cavitation enhancing agent. Furthermore, the enhanced local interaction of a microbubbles and phase-change cavitation enhancing agent mixture can provide a favorable environment for bubble nucleation by subsequent high-pressure (peak negative pressure >5 MPa) pulses, which will further contribute to the clot disruption.

This addition of microbubbles to the phase-change cavitation enhancing agent can be done prior to administration into the patient or can be done within a patient, as both the microbubbles and phase-change cavitation enhancing agents are administered directly into a vein or administered via a catheter with a hollow lumen.

In one implementation, a timing mechanism controls the timing of the ultrasound pulses, and a pulse generator controls the amplitude, frequency, and duration of the ultrasound pulses. The timing mechanism modulates the ultrasound pulses so that there is sufficient time for the cavitation enhancing agent to flow to the blood clot and/or penetrate the clot. Thus, initially, there is a period after administration of a cavitation enhancing agent into the vasculature of a subject during which no cavitation enhancing ultrasound energy is applied. Monitoring ultrasound energy having a power level lower than that needed to enhance cavitation may be applied to monitor the location of the cavitation enhancing agent with respect to the blood clot. A method for estimating an optimal duration of the no-cavitation-enhancing ultrasound period will be described below. Then, the timing mechanism triggers the pulse generator to provide a first ultrasound pulse of a first amplitude, frequency, and duration which causes particles of the phase-change cavitation enhancing agent to convert to microbubbles.

After converting the particles to microbubbles, the timing mechanism then triggers the pulse generator to provide a second pulse of ultrasound energy of a second amplitude, frequency, and duration to cause the microbubbles to oscillate and fragment, facilitating disruption of the blood clot. In one implementation, the first pulse of ultrasound energy comprises a short duration pulse with an amplitude and frequency sufficient to cause a phase change of the phase-change cavitation enhancing agent from liquid to gas, and the second pulse of ultrasound energy at the second frequency and amplitude is a longer duration pulse sufficient to oscillate and/or fragment the microbubbles and cause clot disruption. In one implementation the first amplitude and frequency and second amplitude and frequency are the same, and both sufficient to cause both phase change of the phase-change cavitation enhancing agent and also microbubble oscillation and/or fragmentation. The duration of the applied ultrasound energy which can cause the phase-change cavitation enhancing agent to convert from a liquid droplet to a gas microbubble is typically short, less than 100 cycles or several short bursts not exceeding 100 cycles in total, whereas the duration of ultrasound which causes a bubble to oscillate and/or fragment and disrupt a blood clot is typically longer, greater than 100 cycles, often thousands of cycles, either as a single bursts or several bursts.

Furthermore, application of a longer pulse also increases acoustic radiation force after the bubbles are generated, which pushes the microbubbles and other cavitation enhancing agents (i.e., the particles that have not yet converted to microbubbles) along the ultrasound wave propagation direction, which can be towards the clot if the ultrasound is directed towards the clot, or deeper within the clot if the particles are already within the clot. This will improve the effectiveness of clot lysis due to the cavitation enhancing agents.

Frequencies in the hundred kilohertz to low MHz (100 kHz-2 MHZ) range can be used for both causing phase change of the phase-change cavitation enhancing agent, radiation force to push the cavitation agents in the direction of ultrasound propagation, and oscillation and fragmentation of microbubbles.

Amplitudes required to cause phase conversion of phase-change cavitation enhancing agents depend on the composition of the agent as well as the diameter of the particles, and hence can be tailored to the application, but are typically in the 200 kPa-10 MPa range. Amplitudes required to cause microbubble oscillation and/or fragmentation to result in clot disruption can also be in the 200 kPa-10 MPa range. There is a desire to use as low of an amplitude as possible to minimize ultrasound bioeffects to healthy tissue, so a pulsing sequence that uses a short duration high amplitude pulse to cause the phase change of the phase-change cavitation enhancing agent followed by a longer duration medium amplitude acoustic pulse to oscillate and/or fragment the bubbles would be desirable for clot disruption with minimal effects to surrounding healthy tissue.

The application of the first ultrasound pulse should begin after the phase-change cavitation enhancing agent has time to travel from its administration point (such as the end of the catheter lumen) to the clot. Thus, a time delay can be estimated by the velocity at which the phase-change cavitation enhancing agent is anticipated to travel within the blood vessel and the distance that the phase-change cavitation enhancing agent is expected to travel. The velocity at which the phase-change cavitation enhancing agent is anticipated to travel may be dictated by the blood flow rate and/or by the administration rate of the fluid containing the cavitation enhancing agents through the catheter. The distance that the phase-change cavitation enhancing agent is expected to travel may be estimated based on the administration point of the phase-change cavitation enhancing agent in the blood vessel and the location of the clot determined by prior ultrasound or other imaging technique.

The time duration of the first ultrasound pulse period should be long enough to cause the particles of the phase-change cavitation enhancing agent to convert from liquid droplets to microbubbles, typically on the order of microseconds.

The application of the second ultrasound pulse should commence after a plurality of particles of the phase-change cavitation enhancing agent have converted from liquid droplets to microbubbles. The time duration of the second ultrasound pulse should be long enough to cause the resulting microbubbles to oscillate and/or fragment, which will have an effect of breaking up part of the blood clot, typically on the order of hundreds of microseconds or longer. Although the ultrasound frequency and amplitude of the first ultrasound pulse can be the same as the ultrasound frequency and amplitude of the second ultrasound pulse, in one implementation, the duration of the first ultrasound pulse will be shorter than the duration of the second ultrasound pulse.

After the microbubbles have been made to oscillate and/or fragment, breaking up part of the blood clot, the cycle should be repeated again starting administration of new particles of the phase-change cavitation enhancing agent into the vasculature of the patient, followed by a period of no ultrasound, allowing fresh cavitation enhancing agents to again reach the blood clot and the region around the blood clot. After the period of no cavitation-enhancing ultrasound, the first and second pulses of ultrasound energy are applied to the phase-change cavitation enhancing agent using an ultrasound transducer. This complete cycle is likely repeated many (>10) times, ideally until the blood clot is partially or entirely broken up or dissolved.

In one implementation, ultrasound energy scattered through oscillation and fragmentation of the microbubbles is detected (e.g., by the transmitting ultrasound transducer or an ultrasound transducer that is separate from the transmitting transducer), amplified by a receiver, and utilized to provide feedback to the location of the microbubble oscillation and fragmentation occurring. This may help the physician ensure that the clot disruption is proceeding as intended by indicating the presence, magnitude, and location of the cavitation. In one implementation this feedback is provided in the form of an ultrasound image to the user to allow the user to change the timing of the ultrasound pulses or the timing or rate of infusing of the phase-change cavitation enhancing agent. In another example, the feedback may be provided as a control signal that is routed to the ultrasonic pulser module and/or the infusion pump to change the amount and/or timing of ultrasound energy and/or the amount and/or timing of the cavitation enhancing agent that is provided to affect the clot. The control signal may be used to automatically control the application of ultrasound and/or the timing of infusion of the cavitation enhancing agent until the clot is disrupted by a configurable amount.

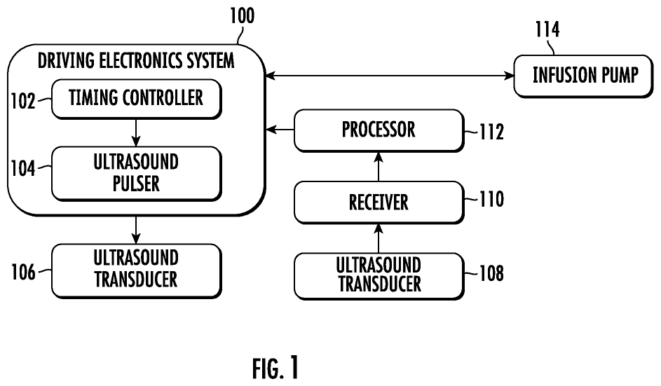

FIG. 1 illustrates one example of a driving electronics system 100 which may comprise a timing controller 102 and an ultrasound pulser 104. Driving electronics system 100 may be integrated with an ultrasound transducer 106. Driving electronics system 100 dictates the time delay between administration of a cavitation enhancing agent and application of cavitation enhancing ultrasound. Driving electronics system 100 may also control the application of the monitoring ultrasound used to determine when the cavitation enhancing agent has reached a blood clot and to determine the location of the blood clot. Driving electronics system 100 may also dictate the frequency, amplitude, and duty cycle of the one or more ultrasound periods used for detecting the location of a blood clot, determining when the cavitation enhancing agent has reached the blood clot, and changing the phases of particles of the cavitation enhancing agent, causing the particles to form bubbles and burst and thereby disrupting the clot. Driving electronics system 100 energizes ultrasound transducer 106, which sends ultrasound pulses to the cavitation enhancing agent or agents. In some implementations, an ultrasound transducer 108, either the same, or separate from ultrasound transducer 106, detects the acoustic energy from particles of the cavitation enhancing agents oscillating and/or fragmenting. This signal is amplified with a receiver 110 and processed by a processor 112. The signal may be converted to an image for guiding the medical procedure and/or may be directly fed back to driving electronics system 100 via a feedback loop that controls the timing, frequency, amplitude, and/or duty cycle of the ultrasound energy generated by ultrasound transducer 106. In one implementation, an infusion pump 114 provides the cavitation enhancing agents into the patient, and the operation of infusion pump 114 may be electronically or manually synchronized with the activation of driving electronics system 100 to administer the ultrasound after, before, or at the same time as the infusion of the cavitation enhancing agent into the patient. In another implementation, the cavitation enhancing agent may be injected into a blood vessel of the patient using a syringe.

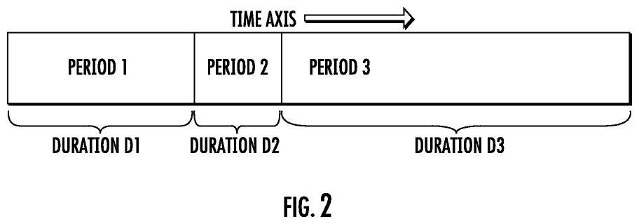

FIG. 2 is a timing diagram showing the relationship of periods 1, 2, and 3. The first period is without cavitation-enhancing ultrasound (but may include monitoring ultrasound) and allows the cavitation enhancing agents to travel to the blood clot. The time duration D1) of this period is dictated by the time it is estimated and/or determined through monitoring for the cavitation enhancing agents (with cavitation enhancing ultrasound off) to either 1) flow from the end of the hollow catheter lumen to the surface and/or into the blood clot, or 2) in the case where a hollow catheter lumen is not used, for example, if the cavitation enhancing agent is injected into a vein using a syringe, the time for a plurality of cavitation agents to refill the area surrounding the clot. In each case, it is desired for a plurality of the cavitation agents to surround and/or penetrate the clot. In one example, the time for particles of the cavitation enhancing agent to reach the clot may be estimated based on the location where the cavitation enhancing agent is administered into the vein, the location of the blood clot (determined in advance of injection or infusion of the cavitation enhancing agent using monitoring ultrasound), the fluid flow rate of the cavitation enhancing agent in the vein in which the clot is located, and, for the catheter administration case, the length of the catheter and the fluid flow rate of the cavitation enhancing agent through the catheter. The fluid flow rates of the cavitation enhancing agent in the vein and/or the catheter may be estimated and/or measured, e.g., monitored using low energy ultrasound. For example, the fluid flow rate in the catheter may be estimated based on the diameter of the catheter, applied pressure, and viscosity of the cavitation enhancing agent. The fluid flow rate of the cavitation enhancing agent in the vein may be estimated using blood vessel radius, viscosity of the cavitation enhancing agent, and the amount that the blood vessel is occluded by the clot. The flow rate of the cavitation enhancing agent in the vein may be monitored by transmitting low energy ultrasound pulses into the vein, detecting ultrasound energy scattered by the cavitation enhancing agent at different locations in the vein, measuring the travel time of the cavitation enhancing agent between the locations, and calculating flow rate based on the distance between monitored locations and the travel time.

After the particles of the phase-change cavitation enhancing agent have surrounded and/or penetrated the clot, period 2 begins. Period 2 is when the ultrasound is activated at a frequency, amplitude, and duty cycle to cause a plurality of the particles of the cavitation enhancing agent to convert from liquid droplets to microbubbles. The ultrasound with frequency, amplitude, and duty cycle to cause a plurality of the particles of the cavitation enhancing agent to convert from liquid droplets to microbubbles is referred to herein as cavitation enhancing ultrasound. The duration D2) dictates the time required to convert a portion of the particles of the cavitation enhancing agents to microbubbles. The third period is also a period where ultrasound is delivered, but the frequency, amplitude, and duty cycle may be different (or the same) from that of period 2 to further optimize the effect of radiation force, bubble oscillation, and bubble fragmentation to cause clot disruption. After period 3, the cycle repeats back to period 1 until the physician decides that the clot is sufficiently disrupted or otherwise chooses to end the procedure.

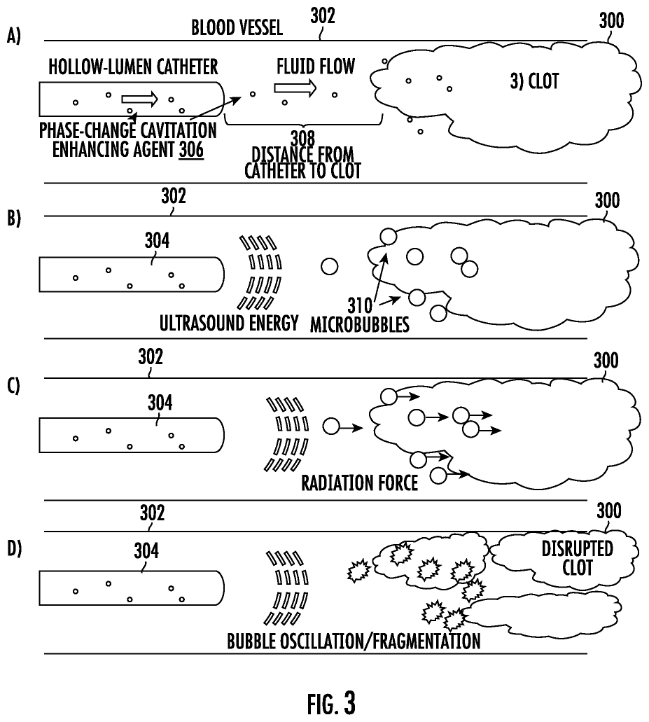

FIG. 3 is a diagram illustrating events in the process. In FIG. 3, in the initial step A), a clot 300 resides within a blood vessel 302 in a patient. In the shown implementation, a hollow lumen catheter 304 is placed into blood vessel 302 in close proximity to clot 300, preferably within 2 cm. A cavitation enhancing agent 306 is administered through catheter 304 towards clot 300. The time that it takes for cavitation enhancing agent 306 to travel from the catheter tip to clot 300, across distance 308 is a function of the fluid flow, which is further a function of blood flow in the vessel and/or the flow rate of fluid being pumped through catheter 304. In this initial step, cavitation enhancing ultrasound is not administered of sufficient energy to cause particles of the phase-change cavitation enhancing agent to convert to microbubbles (low-amplitude ultrasound may be used further guidance, either via an image displayed to the user or an electronic feedback control signal).

After waiting for the estimated time delay determined based on the fluid flow rates and/or monitoring described above, the particles of the phase-change cavitation enhancing agent have reached clot 300 and optimally, have penetrated clot 300. Then, in the second step B) a first frequency, amplitude, and duration of ultrasound are directed toward clot 300, either from a transducer which is part of the catheter or from another location. This ultrasound causes the particles of the phase-change cavitation enhancing agent to convert from liquid droplets to microbubbles 310.

A third phase, depicted by C) and D) shows effects mediated by a second ultrasound frequency and amplitude. These include radiation force which pushes the microbubbles towards or deeper into clot 300 and also microbubble oscillation and/or fragmentation surrounding and within clot 300. The microbubble oscillation and/or fragmentation disrupt blood clot 300.

After a period of microbubble oscillation and/or fragmentation, the cycle is repeated to refresh the clot area with new particles of the phase-change cavitation enhancing agent.

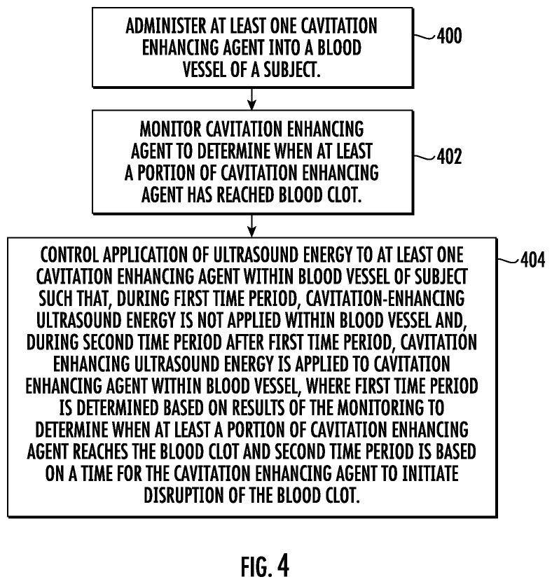

FIG. 4 is a flow chart illustrating an exemplary process for disrupting a blood clot with ultrasound and at least one cavitation enhancing agent. Referring to FIG. 4, in step 400, the process includes administering at least one cavitation enhancing agent into a blood vessel of a subject. For example, a solution of phase-change nanodroplets, microbubbles, or both may be infused or injected into the blood vessel of a subject using an infusion pump or a syringe. The cavitation enhancing agent in one example may be infused through the hollow lumen of a catheter that is integrated with or separate from the ultrasound transducer.

In step 402, the process includes monitoring the at least one cavitation enhancing agent to determine when at least a portion of the at least one cavitation enhancing agent has reached a blood clot. In one example, monitoring the at least one cavitation enhancing agent includes transmitting ultrasound energy into the blood vessel, detecting ultrasound energy scattered by the cavitation enhancing agent, and determining a location of the cavitation enhancing agent within the blood vessel based on the detected ultrasound energy. The ultrasound energy used to monitor the location of the cavitation enhancing agent may, in one example, be at a power level selected so as not to cause a phase change in particles of the cavitation enhancing agent.

In another example, the ultrasound energy used to monitor the location of the cavitation enhancing agent may be transmitted at a power level selected to cause a phase change in particles of the cavitation enhancing agent from a liquid phase to a gaseous phase and form bubbles and to cause the bubbles to burst. The bursting of the bubbles causes scattering of the ultrasound energy, which is detected by the receiver. If the location of the bursting bubbles is determined to be upstream from the clot, the process may include ceasing transmission of the ultrasound energy for a time period estimated for the phase-change cavitation enhancing agent to reach the clot and restarting transmission of the ultrasound energy after the estimated time period for the cavitation enhancing agent to reach the clot.

In either of the examples mentioned in the preceding two paragraphs, results of the monitoring may be used as a feedback control signal to trigger application of the cavitation enhancing ultrasound energy to the cavitation enhancing agent within the blood vessel of the subject. Results of monitoring the cavitation enhancing agent in the blood vessel can be used in combination with the above-described methods for estimating the location of the cavitation enhancing agent to determine when a portion of the cavitation enhancing agent has reached the blood clot and trigger application of the cavitation-enhancing ultrasound energy.

In step 404, the process includes controlling application of ultrasound energy to the at least one cavitation enhancing agent within the blood vessel of the subject such that, during a first time period, cavitation-enhancing ultrasound energy is not applied to the at least one cavitation enhancing agent within the blood vessel and, during a second time period after the first time period, cavitation enhancing ultrasound energy is applied to the at least one cavitation enhancing agent within the blood vessel, wherein the first time period is based on results of the monitoring to determine when the at least a portion of the at least one cavitation enhancing agent reaches the blood clot and the second time period is based on a time for the at least one cavitation enhancing agent to initiate disruption of the blood clot. For example, once the cavitation enhancing agent is determined to have reached the clot, ultrasound energy of a sufficient power level to cause the cavitation enhancing agent to oscillate in diameter and fragment is applied using an ultrasound transducer, which may be the same or a separate transducer from the transducer used to monitor the location of the cavitation enhancing agent. If the cavitation enhancing agent is a phase-change cavitation enhancing agent, the ultrasound transducer may apply ultrasound energy at a power level sufficient to cause particles of the phase-change cavitation enhancing agent to change phase from a liquid phase to a gaseous phase, form bubbles, oscillate in diameter, and burst within or near the clot, thereby disrupting the clot.

### EXAMPLES

The following examples illustrate variations of the subject matter described herein:

In one example, a method of disrupting a blood clot with ultrasound and a phase-change cavitation enhancing agent is provided. According to the method, particles of the phase-change cavitation enhancing agent are administered into the blood of the patient and where a timing mechanism is utilized in order to modulate the ultrasound energy so that there are two time periods. The two time periods include a first time period without ultrasound energy which allows the cavitation agent to flow towards the clot and reach the clot surface. A plurality of particles of the phase-change cavitation agent penetrates into the clot. The two time periods further include a second time period where the ultrasound energy is provided in order that a predetermined portion of the particles of the phase-change cavitation enhancing agent convert to microbubbles. Continued administration of ultrasound energy causes the microbubbles to oscillate in diameter and fragment around and/or within the clot, facilitating disruption of the clot. The cycle of the first and second (or vice versa) time periods are repeated multiple times while the cavitation enhancing agent is introduced into the patient, until the blood clot is partially or entirely disrupted.

In one example, the phase-change cavitation enhancing agent is administered into the blood of the patient via the lumen of a hollow catheter inserted into the vasculature.

In another example, the phase-change cavitation enhancing agent comprises a low-boiling point perfluorocarbon, such as perfluorobutane or perfluoropropane.

In another example, the phase-change cavitation enhancing agent comprises particles of a low-boiling point perfluorocarbon with a boiling point less than 25° C. at atmospheric pressure, in metastable form, which remains in liquid phase at 25° C. and atmospheric pressure.

In another example, particles of the cavitation enhancing agent are less than 300 nm in diameter.

In another example, a plurality of microbubbles is added to the solution containing the phase-change cavitation enhancing agent prior to delivery to the patient.

In another example, the microbubbles in the preceding paragraph comprise a gas core and an encapsulating shell comprising a lipid, protein, or polymer.

In another example, a thrombus-disrupting chemical, such as TPA (Tissue Plasminogen Activator), is also combined with the phase-change cavitation enhancing agent.

In another example, prior to administration into the blood vessel of the patient, the particles of the phase change cavitation enhancing agent are put into a metastable state by being cooled to below 0° C. and exposed to a pressure of greater than one atmosphere.

In another example, the lumen of a hollow catheter is first positioned to within 2 cm of the thrombus prior to administration of the phase-change cavitation enhancing agent.

In yet another example, an ultrasound transducer is located on part of the catheter, which also administers the phase-change cavitation enhancing agent and is positioned within the blood vessel.

In yet another example, an ultrasound transducer is located external to the patient's body, yet is focused and positioned to deliver the ultrasound to the location of the clot.

In yet another example, a pump is used to propel the phase-change cavitation enhancing agent at a constant velocity through the lumen of the hollow catheter and to the site of the blood clot.

In yet another example, activation of the ultrasound source is controlled relative to the activation of the pump which propels the phase change cavitation enhancing agent.

In yet another example, the first period, without ultrasound energy, is of a time duration which is required for a plurality of particles of the cavitation enhancing agent to travel from the administration site into the patient (i.e., from the end of the catheter lumen or the injection site into the vasculature) to the location of the clot.

In another example, ultrasound energy from the microbubbles oscillating and/or fragmenting is detected by one or more ultrasound transducers and utilized to provide feedback to system.

In another example, a method of disrupting a blood clot with ultrasound and a phase-change cavitation enhancing agent is provided. Particles of the phase-change cavitation enhancing agent are administered into the blood of the patient. A timing mechanism is utilized to modulate the ultrasound energy so that there are three time periods. The three time periods include a first time period without ultrasound energy which allows the cavitation agent to flow towards the clot and reach the clot surface. A plurality of the particles of the phase-change cavitation enhancing agent penetrates into the clot. The three time periods include a second time period where the ultrasound energy at a first amplitude, frequency, and duration is provided in order that at least a portion of the particles of the phase-change cavitation enhancing agent convert to microbubbles. The three time periods further include a third time period where administration of ultrasound energy at a second amplitude, frequency, and duration causes at least a portion of the microbubbles to oscillate in diameter and fragment around and/or within the clot, facilitating disruption of the clot. The cycle of the first, second, and third time periods are repeated while the phase-change cavitation enhancing agent is introduced into the patient, until the blood clot is partially or entirely disrupted.

In another example, the ultrasound at the first amplitude, frequency, and duration are sufficient to cause phase transition of the particles of the cavitation enhancing agent from a liquid to a gas

In another example, the ultrasound at the second amplitude, frequency, and duration are sufficient to cause the microbubbles generated from the cavitation enhancing agent to oscillate and fragment, and disrupt the blood clot.

In another example, ultrasound energy from the microbubbles oscillating and/or fragmenting is detected by one or more ultrasound transducers and utilized to provide feedback to system.

Any of the examples described herein can be combined with each other without departing from the scope of the subject matter described herein.

It will be understood that various details of the subject matter described herein may be changed without departing from the scope of the subject matter described herein. Furthermore, the foregoing description is for the purpose of illustration only, and not for the purpose of limitation, as the subject matter described herein is defined by the claims as set forth hereinafter.

What is claimed is:

1. A method for disrupting a blood clot with ultrasound and at least one cavitation enhancing agent, the method comprising:
administering at least one cavitation enhancing agent into a blood vessel of a subject; and
monitoring the at least one cavitation enhancing agent to determine when at least a portion of the at least one cavitation enhancing agent has reached a blood clot; and
controlling application of ultrasound energy to the at least one cavitation enhancing agent within the blood vessel of the subject such that, during a first time period, cavitation-enhancing ultrasound energy is not applied to the at least one cavitation enhancing agent within the blood vessel and, during a second time period after the first time period, cavitation enhancing ultrasound energy is applied to the at least one cavitation enhancing agent within the blood vessel, wherein the first time period is results of the monitoring to determine when the at least a portion of the at least one cavitation enhancing agent reaches the blood clot and the second time period is based on a time for the at least one cavitation enhancing agent to initiate disruption of the blood clot.

2. The method of claim 1 wherein administering the at least one cavitation enhancing agent includes administering a mixture of nanodroplets and microbubbles of the at least one cavitation enhancing agent into the blood vessel.

3. The method of claim 1 wherein administering the at least one cavitation enhancing agent includes separately administering nanodroplets and microbubbles of the at least one cavitation enhancing agent into the blood vessel.

4. The method of claim 1 wherein monitoring the at least one cavitation enhancing agent includes transmitting ultrasound energy into the blood vessel, detecting ultrasound energy scattered by the at least one cavitation enhancing agent, and determining a location of the at least one cavitation enhancing agent within the blood vessel based on the detected ultrasound energy.

5. The method of claim 4 wherein transmitting ultrasound energy into the blood vessel includes transmitting ultrasound energy at a power level selected so as not to cause a phase change in particles of the at least one cavitation enhancing agent.

6. The method of claim 4 wherein transmitting ultrasound energy into the blood vessel includes transmitting ultrasound energy at a power level selected to cause a phase change in particles of the at least one cavitation enhancing agent from a liquid phase to a gaseous phase and form bubbles and to cause the bubbles to burst and wherein monitoring the at least one cavitation enhancing agent includes detecting ultrasound energy scattered by the bursting of the bubbles.

7. The method of claim 6 wherein the first time period ends if a detected location of the bursting of the bubbles is determined to correspond to a location of the blood clot.

8. The method of claim 6 comprising ceasing transmission of the ultrasound energy and extending the first time period if a detected location of the bursting of the bubbles is determined to be upstream from a location of the blood clot.

9. The method of claim 1 wherein the first time period is estimated based on an estimated flow rate of the at least one cavitation enhancing agent through the blood vessel.

10. The method of claim 1 wherein administering the at least one cavitation enhancing agent into the blood vessel includes administering the at least one cavitation enhancing agent via a hollow lumen of a catheter and the first time period is based on an estimated flow rate of the at least one cavitation enhancing agent through the hollow lumen.

11. The method of claim 1 wherein the at least one cavitation enhancing agent comprises nanodroplets and the second time period is based on a time for a portion of the nanodroplets to convert to microbubbles and oscillate in diameter.

12. The method of claim 11 wherein controlling the application of the ultrasound energy comprises applying the cavitation enhancing ultrasound energy to the at least one cavitation enhancing agent for a third time period after the second time period, wherein the third time period is based on a time for a portion of the microbubbles to fragment.

13. The method of claim 1 comprising monitoring results of application of the cavitation enhancing ultrasound energy to the at least one cavitation enhancing agent and wherein controlling application of the ultrasound energy includes controlling the application based on the results.

14. The method of claim 13 wherein monitoring results of the application of the cavitation enhancing ultrasound energy includes using an ultrasound transducer to monitor ultrasound energy scattered by the at least one cavitation enhancing agent proximal to the blood clot, generating an image of the scattered ultrasound energy, and displaying the image to a user.

15. The method of claim 13 wherein monitoring results of the application of the cavitation enhancing ultrasound energy includes generating a feedback control signal based on ultrasound energy scattered by the at least one cavitation enhancing agent and automatically controlling at least one of the administering of the at least one cavitation enhancing agent and the application of the cavitation enhancing ultrasound energy using the feedback control signal.

16. A system for disrupting a blood clot with ultrasound and at least one cavitation enhancing agent, the system comprising:
at least one cavitation enhancing agent capable of being administered into a blood vessel of a subject;
a receiver for monitoring the at least one cavitation enhancing agent to determine when at least a portion of the at least one cavitation enhancing agent has reached a blood clot; and
a timing controller for controlling application of ultrasound energy to the at least one cavitation enhancing agent within the blood vessel of the subject such that, during a first time period, cavitation-enhancing ultrasound energy is not applied to the at least one cavitation enhancing agent within the blood vessel and, during a second time period after the first time period, cavitation enhancing ultrasound energy is applied to the at least one cavitation enhancing agent within the blood vessel, wherein the first time period is based on results of the monitoring to determine when the at least a portion of the at least one cavitation enhancing agent reaches the blood clot and the second time period is based on a time for the at least one cavitation enhancing agent to initiate disruption of the blood clot.

17. The system of claim 16 wherein the at least one cavitation enhancing agent comprises a mixture of nanodroplets and microbubbles.

18. The system of claim 16 wherein the at least one cavitation enhancing agent comprises nanodroplets and microbubbles separately administerable into the blood vessel.

19. The system of claim 16 comprising an ultrasound transducer integrated with or separate from the receiver for transmitting ultrasound energy into the blood vessel, wherein the receiver is configured to detect ultrasound energy scattered by the at least one cavitation enhancing agent and the timing controller is configured to determine a location of the at least one cavitation enhancing agent within the blood vessel based on the detected ultrasound energy.

20. The system of claim 19 wherein the ultrasound transducer is configured to transmit the ultrasound energy at a power level selected so as not to cause a phase change in particles of the at least one cavitation enhancing agent.

21. The system of claim 19 wherein the ultrasound transducer is configured to transmit the ultrasound energy at a power level selected to cause a phase change in particles of the at least one cavitation enhancing agent from a liquid phase to a gaseous phase and form bubbles and to cause the bubbles to burst and the receiver is configured to detect the ultrasound energy scattered by the bursting of the bubbles.

22. The system of claim 21 wherein the first time period ends if a detected location of the bursting of the bubbles is determined to correspond to a location of the blood clot.

23. The system of claim 21 wherein the timing controller is configured to cease transmission of the ultrasound energy and extend the first time period if a detected location of the bursting of the bubbles is determined to be upstream from a location of the blood clot.

24. The system of claim 16 comprising a catheter having a hollow lumen through which the at least one cavitation enhancing agent is administerable and the first time period is based on an estimated flow rate of the at least one cavitation enhancing agent through the hollow lumen.

25. The system of claim 16 wherein the at least one cavitation enhancing agent comprises nanodroplets and the second time period is based on a time for a portion of the nanodroplets to convert to microbubbles and oscillate in diameter.

26. The system of claim 25 wherein the timing controller is configured to control the application of the ultrasound energy by applying the cavitation enhancing ultrasound energy to the at least one cavitation enhancing agent for a third time period after the second time period, wherein the third time period is based on a time for a portion of the microbubbles to fragment.

27. The system of claim 26 wherein the receiver is configured to monitor ultrasound energy scattered by the at least one cavitation enhancing agent proximal to the blood clot, generate an image of the scattered ultrasound energy, and display the image to a user.

28. The system of claim 26 wherein the receiver is configured to generate a feedback control signal based on ultrasound energy scattered by the at least one cavitation enhancing agent and the timing controller is configured to automatically control at least one of the administering of the at least one cavitation enhancing agent and the application of the cavitation enhancing ultrasound energy using the feedback control signal.

29. A non-transitory computer readable medium having stored thereon computer-executable instructions that when executed by a processor of a computer control the computer to implement the method of any one of claims 1-15.
